(19) United States
(12) Patent Application Publication
(10) Pub. No.: US 2020/0138935 A1
(43) Pub. Date: May 7, 2020

(54) CHLAMYDIA NANOEMULSION VACCINE

(71) Applicant: NanoBio Corporation, Ann Arbor, MI (US)

(72) Inventors: Ali FATTOM, Rockville, MD (US); Vira BITKO, Ann Arbor, MI (US)

(21) Appl. No.: 16/630,679

(22) PCT Filed: Jul. 13, 2018
(86) PCT No.: PCT/US2018/041955

§ 371 Date: Jan. 13, 2020

Related U.S. Application Data
(60) Provisional application No. 62/532,348, filed on Jul. 13, 2017.

(51) Int. Cl. A61K 39/118; A61K 9/107; C07K 14/295; A61K 9/00

(52) U.S. Cl. CPC A61K 39/118; A61K 9/107; A61K 2039/521; A61K 9/0029; C07K 14/295

(57) ABSTRACT

The present application relates to the field of human immunology, in particular, a chlamydia vaccine. The subunit vaccine composition may comprise isolated antigens from chlamydia bacteria, fusion proteins or fragments thereof, live or attenuated bacteria, or other bacterial components mixed in varied combination with a nanoemulsion, which provides a potent immune enhancer.

### CROSS-REFERENCE TO RELATED APPLICATION

The present application claims priority to U.S. Provisional Application No. 62/532,348, filed on Jul. 13, 2018, the contents of which are specifically incorporated by reference.

### FIELD OF THE INVENTION

The present application relates to the field of human immunology, in particular, a Chlamydia vaccine, or more specifically a Chlamydia trachomatis vaccine.

### BACKGROUND OF THE INVENTION

### I. Background Regarding Chlamydia Infections

Chlamydial organisms cause a wide spectrum of diseases in humans, mammals, and birds, and the resultant infections have an enormous economic impact on both human and animal health and on agricultural industries worldwide. The two principal human pathogens are Chlamydia trachomatis and Chlamydophila pneumonia, although many other species and serovars that effect various animals also exist. Chlamydia trachomatis causes chronic conjunctivitis and it is also the most common cause of sexually transmitted disease in humans. Infections often go unnoticed and, if left untreated, can ultimately result in scarring and fibrosis of ocular and genital tissues, resulting in trachoma (the leading cause of preventable blindness worldwide) and pelvic inflammatory disease, which can lead to infertility, ectopic pregnancy and chronic pelvic pain. Chlamydophila pneumonia causes acute respiratory disease and is responsible for 5-10% of the cases of community-acquired pneumonia, bronchitis and sinusitis. The organism has also been associated with chronic obstructive pulmonary disease, asthma, reactive airway disease, Reiter's syndrome, sarcoidosis and atherosclerosis. Thus, chlamydia infections represent a major public health concern.

Yet currently, there is no vaccine against chlamydia. Despite the availability of antibiotics, chlamydia remains a significant global health problem, and the consequences of lack of treatment can be devastating in terms of public health and safety as well as to industry and the economy.

### II. Background Regarding Nanoemulsions

The lack of an adequate vaccine for chlamydia prompted the inventors to elaborate on previous findings regarding the novel features of a nanoemulsion as an immune enhancer for antigens. A nanoemulsion, while providing an adjuvant effect, also helps in antigen presentation by attracting the appropriate cell types and activating multiple arms of the immune response. (See, for instance, Hamouda et al., Efficacy, immunogenicity and stability of a novel intranasal nanoemulsion adjuvanted influenza vaccine in a murine model. HUM VACCINE. 2010. 6:585-594; Bielinska et al., Induction of Th17 cellular immunity with a novel nanoemulsion adjuvant. CRIT REV IMMUNOL. 2010. 30:189-199; Makidon et al., Pre-clinical evaluation of a novel nanoemulsion-based hepatitis B mucosal vaccine. PLOS-ONE. 2008. 3:e2954).

As with most vaccines, greater immunogenicity is also sought as it correlates with greater efficacy in both humans and animals. The prior art has typically disclosed the use of recombinant proteins (e.g., U.S. Pat. Nos. 7,192,595; 6,194,546; 5,962,298), as well as the addition of adjuvants such as aluminum (U.S. Pat. No. 6,861,244) and muramyldipeptide (U.S. Pat. No. 4,826,687) to compositions to increase the immunogenicity. However, there still exists a need to develop highly effective chlamydia vaccines with improved storage stability and ease of administration, which are characteristics of the nanoemulsion vaccines of the present invention.

Prior teachings related to nanoemulsions are described in U.S. Pat. No. 6,015,832, which is directed to methods of inactivating Gram-positive bacteria, a bacterial spore, or Gram-negative bacteria. The methods comprise contacting the Gram-positive bacteria, bacterial spore, or Gram-negative bacteria with a bacteria-inactivating (or bacterial-spore inactivating) emulsion. U.S. Pat. No. 6,506,803 is directed to methods of killing or neutralizing microbial agents (e.g., bacterial, virus, spores, fungus, on or in humans using an emulsion. U.S. Pat. No. 6,559,189 is directed to methods for decontaminating a sample (human, animal, food, medical device, etc.) comprising contacting the sample with a nanoemulsion. The nanoemulsion, when contacted with bacteria, virus, fungi, protozoa or spores, kills or disables the pathogens. The antimicrobial nanoemulsion comprises a quaternary ammonium compound, one of ethanol/glycerol/PEG, and a surfactant. U.S. Pat. No. 6,635,676 is directed to two different compositions and methods of decontaminating samples by treating a sample with either of the compositions. Composition 1 comprises an emulsion that is antimicrobial against bacteria, virus, fungi, protozoa, and spores. The emulsions comprise an oil and a quaternary ammonium compound. U.S. Pat. No. 7,314,624 is directed to methods of inducing an immune response to an immunogen comprising treating a subject via a mucosal surface with a combination of an immunogen and a nanoemulsion. The nanoemulsion comprises oil, ethanol, a surfactant, a quaternary ammonium compound, and distilled water. US-2005-0208083 and US-2006-0251684 are directed to nanoemulsions having droplets with preferred sizes. US-2007-0054834 is directed to compositions comprising quaternary ammonium halides and methods of using the same to treat infectious conditions. The quaternary ammonium compound may be provided as part of an emulsion. US-2007-0036831 and US 2011-0200657 are directed to nanoemulsions comprising an anti-inflammatory agent. Other publications that describe nanoemulsions include U.S. Pat. No. 8,226,965 for “Methods of treating fungal, yeast and mold infections;” US 2009-0269394 for “Methods and compositions for treating onychomycosis;” US 2010-0075914 for “Methods for treating herpes virus infections;” US 2010-0092526 for “Nanoemulsion therapeutic compositions and methods of using the same;” US 2010-0226983 for “Compositions for treatment and prevention of acne, methods of making the compositions, and methods of use thereof,” US 2012-0171249 for “Compositions for inactivating pathogenic microorganisms, methods of making the compositions, and methods of use thereof,” and US 2012-0064136 for “Anti-aging and wrinkle treatment methods using nanoemulsion compositions.” However, none of these references teach the methods, compositions, and kits of the present invention.

In particular, U.S. Pat. No. 7,314,624 describes nanoemulsion vaccines. However, this reference does not teach the ability to induce a protective immune response to chlamydia using the immunogens of the invention.

Prior art directed to vaccines includes, for example, U.S. Pat. No. 7,731,967 for “Composition for inducing immune response” (Novartis), which describes an antigen/adjuvant complex comprising at least two adjuvants. U.S. Pat. No. 7,357,936 for “Adjuvant systems and vaccines” (GSK) describes a combination of adjuvant and antigens. U.S. Pat. No. 7,323,182 for “Oil in water emulsion containing saponins” (GSK) describes a vaccine composition with an oil/water formulation. U.S. Pat. No. 6,867,000 for “Method of enhancing immune response to herpes” (Wyeth) describes a combination of viral antigens and cytokines (IL12). U.S. Pat. No. 6,692,752 for “Methods of treating human females susceptible to HSV infection” (GSK) describes a method of treating an HSV 1-/2-female human subject susceptible to herpes simplex virus (HSV) infection. The method comprises administering to the subject an effective amount of a vaccine formulation comprising an adjuvant and an antigen which is or is derived from the group consisting of HSV-1 glycoprotein D, HSV-2 glycoprotein D and an immunological fragment thereof. U.S. Pat. Nos. 6,623,739, 6,372,227, and 6,146,632, all for “Vaccines” (GSK), are directed to an immunogenic composition comprising an antigen and/or antigen composition and an adjuvant consisting of a metabolizable oil and alpha tocopherol in the form of an oil in water emulsion. U.S. Pat. No. 6,451,325 for “Adjuvant formulation comprising a submicron oil droplet emulsion” (Chiron) is directed to an adjuvant composition comprising a metabolizable oil, an emulsifying agent, and an antigenic substance, wherein the oil and emulsifying agent are present in the form of an oil-in-water emulsion. The adjuvant composition does not contain any polyoxypropylene-polyoxyethylene block copolymer; and the antigenic substance is not present in the internal phase of the adjuvant composition.

Finally, US 20040151734 for “Vaccine and method of use” (GSK) describes a method of treating a female human subject suffering from or susceptible to one or more sexually transmitted diseases (STDs). The method comprises administering to a female subject in need thereof an effective amount of a vaccine formulation comprising one or more antigens derived from or associated with an STD-causing pathogen and an adjuvant.

Clearly, there is a need in the art for improved therapies relating to treating, preventing, or curing chlamydia infections in a subject, including humans and animals. Additionally, there remains a need in the art for an effective chlamydia vaccine and methods of making and using the same. The present invention satisfies these needs.

### SUMMARY OF THE INVENTION

The present invention provides a novel approach for inducing a protective immune response against various chlamydia infections. The vaccine can be useful against known strains and serovars of chlamydia. Combining a nanoemulsion with whole chlamydia bacteria (native or mutant) and/or one or more chlamydia antigens, either recombinant or isolated, presents a novel combination that provides for the rational basis of vaccine development for use in humans and animals. The present invention provides compositions and methods for inducing an immune response to chlamydia infection in a subject.

Accordingly, in one aspect, the disclosed invention is directed to a vaccine composition comprising an immune enhancing nanoemulsion, wherein the nanoemulsion comprises an oil-in-water nanoemulsion or a dilution thereof and at least one chlamydia bacteria antigen, wherein the chlamydia bacteria antigen is whole chlamydia virus, an isolated chlamydia antigen, a recombinant chlamydia antigen, or a combination thereof, and wherein the chlamydia bacteria antigen is present within the nanoemulsion. In some embodiments, the chlamydia bacteria antigen is inactivated prior to incorporation into a nanoemulsion, while in others the chlamydia bacteria antigen is inactivated by the nanoemulsion.

In some embodiments, the chlamydia bacteria antigen is derived from chlamydia bacteria and comprises at least one of the following: major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, and CT144 or an immunogenic fragment thereof.

In some embodiments, one or more chlamydia antigens further comprise nucleotide modifications denoting attenuating phenotypes. In other embodiments, at least one chlamydia antigen is present in a fusion protein. In still other embodiments, at least one chlamydia antigen is present in an immunogenic peptide fragment of major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, and CT144 or an immunogenic derivative thereof.

In some embodiments, the immune enhancing nanoemulsion is capable of inducing Th1, Th2, Th17, and/or IFN-γ immune responses.

In some embodiments, the nanoemulsion particle size is from about 300 nm up to about 600 nm, and the composition may additionally comprise an adjuvant and/or a pharmaceutically acceptable carrier. In some embodiments, the vaccine composition is administered either parenterally, intravaginally, orally or intranasally, and when it is parenteral administered it may be by subcutaneous, intraperitoneal or intramuscular injection.

In another aspect, the disclosed invention is directed to, a method for inducing an immune response against infection caused by chlamydia bacteria comprising the step of administering to an individual an effective amount of a nanoemulsion chlamydia bacteria vaccine composition comprising a nanoemulsion, wherein the nanoemulsion comprises an oil-in-water nanoemulsion or a dilution thereof and at least one chlamydia bacteria antigen, wherein the chlamydia bacteria antigen is whole chlamydia, an isolated chlamydia antigen, a recombinant chlamydia antigen, or a combination thereof, and wherein the chlamydia bacteria antigen is present within the nanoemulsion.

In some embodiments, the chlamydia bacteria antigen is derived from chlamydia bacteria and comprising at least one major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, or CT144 or an immunogenic fragment thereof.

In some embodiments, the step of administering comprises parenterally, intravaginally, orally and/or intranasally administering the nanoemulsion.

In another aspect, the disclosed invention is directed to a method for preparing a nanoemulsion chlamydia bacteria vaccine useful for the treatment or prevention of an chlamydia infection in a subject comprising: synthesizing in a eukaryotic host one or more full length or immunogenic fragment chlamydia bacteria antigens utilizing recombinant DNA genetics vectors and constructs, wherein the chlamydia bacteria antigen is selected from the group consisting of major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, and CT144; isolating the one or more antigens or immunogenic fragments thereof from the prokaryotic host; and formulating the one or more antigens with an oil-in-water nanoemulsion.

In some embodiments, the prokaryotic host is an E. coli.

In some embodiments, the chlamydia bacteria is C. trachomatis. And in some embodiments, the subject is a human, while in others, the subject is an animal.

In another aspect, the disclosed invention is directed to a subunit vaccine composition comprising an immune enhancing nanoemulsion combined with one or more chlamydia bacteria antigens, wherein the nanoemulsion further comprises an oil-in-water nanoemulsion or a dilution thereof and isolated bacterial antigens preferentially comprised within the nanoemulsion.

In some embodiments, the one or more antigens are derived from chlamydia bacteria and comprise at least one major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, or CT144 or an immunogenic fragment thereof.

In some embodiments, the subunit vaccine composition may additionally comprise an adjuvant or at least one pharmaceutically acceptable carrier.

In some embodiments, the vaccine composition is administered either parenterally, orally, intravaginally, and/or intranasally and when it is parenteral administered it may be by subcutaneous, intraperitoneal or intramuscular injection.

The foregoing general description and following detailed description are exemplary and explanatory and are intended to provide further explanation of the invention as claimed. Other objects, advantages, and novel features will be readily apparent to those skilled in the art from the following brief description of the drawings and detailed description of the invention.

### BRIEF DESCRIPTION OF THE DRAWINGS

FIG. 1 shows TEM cross section images of the 20% W805EC nanoemulsion with and without 30 μg total HA. FIG. 1A shows a 20% nanoemulsion without added antigen. FIG. 1B (panel on the right) shows a 20% nanoemulsion combined with 30 μg Fluzone®, and illustrates that the HA antigens are located in the oil droplets. The darkly stained antigens are located outside of the nanoemulsion particles.

### DETAILED DESCRIPTION OF THE PREFERRED EMBODIMENTS

The present invention provides a novel approach for inducing a protective immune response against chlamydia infection. Combining a nanoemulsion with chlamydia whole bacteria and/or multiple chlamydia antigens presents a novel combination that provides for the rational basis of vaccine development for use in humans and animals.

### I. General Description of the Invention

The present invention provides composition and methods for enhancement of the immune responses to chlamydia bacteria. Specifically, the present invention provides composition and methods for the use of a nanoemulsion as an immune enhancer and adjuvant to boost and increase the breath of the immune response to chlamydia whole bacteria and/or bacterial antigens. In some embodiments, at least one isolated chlamydia antigen is mixed in varied proportions with a nanoemulsion.

Prior to the present invention, it was observed that the novel broad-based immune enhancement functions of a nanoemulsion results in activation of multiple arms of the immune response to other pathogens and antigens, including influenza, hepatitis B surface antigens, and respiratory syncytial virus. However, unlike influenza and hepatitis B virus, for which current licensed vaccines exist for human use, no current vaccines exist for chlamydia.

The present invention is based on a novel combination of chlamydia whole bacteria and/or antigens combined with a nanoemulsion and is contemplated to provide a robust and comprehensive immune response by inducing Th1, Th2, Th17, and IFN-γ arms of the immune response, which will result in an optimal prophylactic vaccine against chlamydia.

Experiments conducted with influenza and hepatitis B virus (HBV) demonstrated that a nanoemulsion coupled with a single antigen is capable of inducing a protective immune response.

Experiments conducted during the course of the development of the current invention demonstrated that a nanoemulsion added to hepatitis B surface antigen (HBsAg) and administered intranasally was a safe and effective hepatitis B vaccine. The mucosal vaccine induced a Th1 associated cellular immune response, with concomitant neutralizing antibodies production. A single nasal immunization of the HBsAg nanoemulsion mixture produced a rapid induction of serum antibodies that was comparable to currently administered intramuscular vaccines. Further, there was demonstration of affinity maturation in the antibody response, which is predictive of the potential efficacy of vaccine.

Another emerging component of vaccine protective efficacy is the induction of T-helper-17 (Th17) cytokine responses. The demonstration that IL-17 contributes to the normal immune response to pathogens has been further utilized to show relevance in vaccination strategies. In the development of the current invention, mucosal immunization with nanoemulsion can produce adjuvant effects in activating Th1 and Th17 immunity. Mucosal immunization with nanoemulsion resulted in activation of innate immune response which directly helps in the induction of Th1 and Th17 cells. The results further clarify the immune enhancing features of nanoemulsion importance in the field of vaccination for the induction of cellular immunity against pathogens, such as chlamydia. Likewise, it has become widely accepted that T cell driven IFN-γ and Th17 responses are critical for clearing infection.

The present invention provides compositions and methods for enhancement of the immune responses. Specifically, the present invention discloses compositions and methods for the use of nanoemulsions as an immune enhancer, providing adjuvant effects to chlamydia vaccine compositions.

In one embodiment, a chlamydia vaccine composition comprises an immune enhancing nanoemulsion and whole chlamydia bacteria, either native, recombinant, or mutant, wherein the nanoemulsion further comprises an oil-in-water nanoemulsion or a dilution thereof, and wherein the chlamydia bacteria is preferably present within the nanoemulsion.

In another embodiment, subunit vaccines can be constructed with one or more of chlamydia antigens mixed with nanoemulsion. It is entirely possible to have chlamydia antigens, including but not limited to major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, CT144, and fusions, derivatives, or fragments thereof added together and mixed with nanoemulsion in a resulting vaccine composition, as well as chlamydia whole bacteria. It is envisaged that any combination of chlamydia antigens, as well as chlamydia whole bacteria, can be mixed with nanoemulsion to produce a resulting vaccine composition. The vaccine composition can be delivered via intranasal, intravaginal, oral, or other pharmaceutically acceptable route, including other mucosal routes.

In one embodiment, a multivalent subunit vaccine can be constructed utilizing antigens, such as major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, CT144, CopB, CopD, CT584 and fusions, derivatives, or fragments thereof, mixed with nanoemulsion. The antigens can be combined in various combinations to produce an effective vaccine against any desired species or subtype of chlamydia.

The quantities of each component present in the nanoemulsion and/or nanoemulsion vaccine refer to a therapeutic nanoemulsion and/or nanoemulsion chlamydia vaccine.

In one embodiment of the invention, the nanoemulsion chlamydia vaccine comprises at least one chlamydia immunogen and droplets having an average diameter of less than about 1000 nm and: (a) an aqueous phase; (b) about 1% oil to about 80% oil; (c) about 0.1% to about 50% organic solvent; (d) about 0.001% to about 10% of a surfactant or detergent; or (e) any combination thereof. In another embodiment of the invention, the nanoemulsion vaccine comprises at least one chlamydia immunogen and: (a) an aqueous phase; (b) about 1% oil to about 80% oil; (c) about 0.1% to about 50% organic solvent; (d) about 0.001% to about 10% of a surfactant or detergent; and (e) at least one chlamydia immunogen. In another embodiment of the invention, the nanoemulsion lacks an organic solvent.

In a further embodiments, the nanoemulsion comprises a quaternary ammonium-containing compound. The present invention is not limited to a particular quaternary ammonium containing compound. A variety of quaternary ammonium containing compounds are contemplated including, but not limited to, Alkyl dimethyl benzyl ammonium chloride, dialkyl dimethyl ammonium chloride, n-Alkyl dimethyl benzyl ammonium chloride, n-Alkyl dimethyl ethylbenzyl ammonium chloride, Dialkyl dimethyl ammonium chloride, and n-Alkyl dimethyl benzyl ammonium chloride.

In certain embodiments, the nanoemulsion further comprises a cationic halogen containing compound. The present invention is not limited to a particular cationic halogen containing compound. A variety of cationic halogen containing compounds are contemplated including, but not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, and tetradecyltrimethylammonium halides. The present invention nanoemulsion is also not limited to a particular halide. A variety of halides are contemplated including, but not limited to, halide selected from the group consisting of chloride, fluoride, bromide, and iodide.

The nanoemulsion chlamydia vaccine of the invention can be administered to a subject using any pharmaceutically acceptable method, for example, intranasal, buccal, sublingual, oral, rectal, ocular, parenteral (intravenously, intradermally, intramuscularly, subcutaneously, intracisternally, intraperitoneally), pulmonary, intravaginal, locally administered, topically administered, topically administered after scarification, mucosally administered, via an aerosol, or via a buccal or nasal spray formulation.

In yet another embodiment of the invention, the nanoemulsion chlamydia vaccines of the invention are useful in treating and/or preventing a chlamydia infection which is drug resistant. For example the infection can be of a chlamydia resistant to an antibiotic drug such as azithromycin or doxycycline. The emergence of bacterial serovars resistant to commonly used antibiotic drugs is a problem in the clinical setting, particularly in immunocompromised patients. The present invention addresses this problem by alleviating the need for antibiotics to treat chlamydia infection.

The nanoemulsion chlamydia vaccine of the invention can be formulated into any pharmaceutically acceptable dosage form, such as a liquid dispersion, gel, aerosol, pulmonary aerosol, nasal aerosol, ointment, cream, semi-solid dosage form, or a suspension. Additionally, the nanoemulsion chlamydia vaccine may be a controlled release formulation, sustained release formulation, immediate release formulation, or any combination thereof. Further, the nanoemulsion chlamydia vaccine may be a transdermal delivery system such as a patch or administered by a pressurized or pneumatic device.

The immune response of the subject can be measured by determining the titer and/or presence of antibodies against the chlamydia immunogen after administration of the nanoemulsion chlamydia vaccine to evaluate the humoral response to the immunogen. Seroconversion refers to the development of specific antibodies to an immunogen and may be used to evaluate the presence of a protective immune response. Such antibody-based detection is often measured using enzyme-linked immunosorbent (ELISA) assay. Persons of skill in the art would readily select and use appropriate detection methods.

Another method for determining the subject's immune response to the nanoemulsion chlamydia vaccine is to determine the cellular immune response, such as through immunogen-specific cell responses, such as cytotoxic T lymphocytes, or immunogen-specific lymphocyte proliferation assay. Additionally, challenge by the pathogen may be used to determine the immune response, either in the subject, or, more likely, in an animal model. A person of skill in the art would be well versed in the methods of determining the immune response of a subject and the invention is not limited to any particular method.

In another embodiment of the invention, the nanoemulsion chlamydia vaccines of the invention result in the generation of robust neutralizing antibodies. For example, administration of at least one dose, or two, three, four, or five doses of a nanoemulsion chlamydia vaccine according to the invention can result in antibody end point titers or μg/ml (either measurement used for the end point), ranging from 2 to 2×10⁵ or more. For example, antibody titers resulting from the administration of the disclosed nanoemulsion chlamydia vaccines may be about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 or more.

### II. Nanoemulsion Chlamydia Vaccines

A. Chlamydia Immunogen

The chlamydia immunogen present in the nanoemulsion chlamydia vaccines of the invention can be whole chlamydia bacteria, including native, recombinant, and mutant serovars of chlamydia. In one embodiment of the invention, the chlamydia bacteria can be resistant to one or more antibiotic drugs. Any known chlamydia species or serovar can be used in the vaccines of the invention.

Chlamydia, and specifically Chlamydia trachomatis, is a major cause of sexually transmitted disease worldwide. In women, infection of the upper genital tract with chlamydiae is associated with pelvic pain and salpingitis which may lead to infertility or ectopic pregnancy. In large areas of the world C. trachomatis is the major infectious cause of female infertility. The cost of treating chlamydial infections and their sequelae runs to billions of dollars annually for the USA alone. In addition, C. trachomatis is also the world's major cause of preventable blindness (trachoma). An effective chlamydial vaccine is a major public health goal.

Empirical attempts to vaccinate against trachoma by immunization with whole C. trachomatis resulted in evidence of short-term, serovar-specific (homotypic) immunity to ocular chlamydial infection. However, peptides, antigens, and other immunogenic portions or fragments of the bacteria can elicit an immune response as well.

Aside from C. trachomatis, there are numerous other types of chlamydia species and serovars that cause serious human and veterinary infections. The chlamydia genus is composed of obligate intracellular parasites which can infect a variety of hosts. Chlamydia trachomatis is a human pathogen, while Chlamydia suis affects swine, Chlamydia muridarum affects mice and hamsters, and Chlamydia felis affects cats. Additionally, Chlamydophila psittaci is a lethal intracellular bacterial species that may cause endemic avian chlamydiosis, epizootic outbreaks in mammals, and respiratory psittacosis in humans; Chlamydophila pneumoniae infects humans as well as koalas, emerald tree boas (Corallus caninus), iguanas, chameleons, frogs, and turtles, and it is a major cause of pneumonia in human subjects; and Chlamydophila pecorum can cause reproductive disease, infertility, urinary tract disease, and death in cattle, sheep and goats, koalas, and swine. All of the above are included in types of chlamydia that may be treated or prevented by the disclosed vaccines, depending on the immunogen or immunogens included in the vaccine.

Additionally, the chlamydia immunogen present in the nanoemulsion chlamydia vaccines of the invention can be one or more chlamydia antigens, which are include, but are not limited to, major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, and fusions, derivatives, or fragments thereof. Other chlamydial antigens that may be present in the nanoemulsion chlamydia vaccines of the invention include CT111, CT242, CT687, CT823 and CT144.

The chlamydia antigens are generally extracted from bacterial isolates from infected cell cultures, or produced by synthetically, or by using recombinant DNA methods and expressed in E. coli. The chlamydia antigens can be modified by chemical, genetic, or enzymatic means resulting in fusion proteins, peptides, or fragments. The chlamydia antigens can be obtained from any known chlamydia species or serovar, including but not limited to those listed above.

The chlamydia immunogen present in the vaccines of the invention can also be whole chlamydia bacteria combined with one or more chlamydia antigens. In embodiments in which the vaccines comprise whole bacteria, the chlamydia may be live, killed, or attenuated.

Any suitable amount of chlamydia immunogen can be used in the nanoemulsion chlamydia vaccines of the invention. For example, the nanoemulsion chlamydia vaccine can comprise less than about 200 μg of chlamydia immunogen (total chlamydia immunogen and not per chlamydia immunogen). In another embodiment of the invention, the nanoemulsion chlamydia vaccine can comprise less than about 90 less than about 80 less than about 70 less than about 60 less than about 50 less than about 40 less than about 30 less than about 20 less than about 15 less than about 10 less than about 9 less than about 8 less than about 7 less than about 6 less than about 5 less than about 4 less than about 3 less than about 2 or less than about 1 μg of chlamydia immunogen (total chlamydia immunogen and not per chlamydia immunogen).

In another embodiment of the invention, the chlamydia vaccines of the invention comprise about 1.0×10⁵ cfu (colony forming units) up to about 1.0×10⁸ cfu, and any amount in-between, of an chlamydia bacteria or antigen. The chlamydia bacteria or antigen is inactivated by the presence of the nanoemulsion adjuvant. For example, the chlamydia vaccines can comprise about 1.0×10⁵, about 1.1×10⁵, about 1.2×10⁵, about 1.3×10⁵, about 1.4×10⁵, about 1.5×10⁵, about 1.6×10⁵, about 1.7×10⁵, about 1.8×10⁵, about 1.9×10⁵, about 2.0×10⁵, about 2.1×10⁵, about 2.2×10⁵, about 2.3×10⁵, about 2.4×10⁵, about 2.5×10⁵, about 2.6×10⁵, about 2.7×10⁵, about 2.8×10⁵, about 2.9×10⁵, about 3.0×10⁵, about 3.1×10⁵, about 3.2×10⁵, about 3.3×10⁵, about 3.4×10⁵, about 3.5×10⁵, about 3.6×10⁵, about 3.7×10⁵, about 3.8×10⁵, about 3.9×10⁵, about 4.0×10⁵, about 4.1×10⁵, about 4.2×10⁵, about 4.3×10⁵, about 4.4×10⁵, about 4.5×10⁵, about 4.6×10⁵, 4.7×10⁵, 4.8×10⁵, 4.9×10⁵, 5.0×10⁵, 5.5×10⁵, 6.0×10⁵, 6.5×10⁵, 7.0×10⁵, 7.5×10⁵, 8.0×10⁵, 8.5×10⁵, 9.0×10⁵, 9.5×10⁵, 1.0×10⁶, 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, about 9.5×10⁶, about 1.0×10⁷, about 1.5×10⁷, about 2.0×10⁷, about 2.5×10⁷, about 3.0×10⁷, about 3.5×10⁷, about 4.0×10⁷, about 4.5×10⁷, about 5.0×10⁷, about 5.5×10⁷, about 6.0×10⁷, about 6.5×10⁷, about 7.0×10⁷, about 7.5×10⁷, about 8.0×10⁷, about 8.5×10⁷, about 9.0×10⁷, about 9.5×10⁷, or about 1.0×10⁸ cfu of a chlamydia bacteria.

In another embodiment, the chlamydia vaccines of the invention are cross-reactive against at least one other chlamydia serovar or species not present in the vaccine (or cross-reactive against one or more chlamydia serovar or species). As it is known to one of ordinary skill in the art, cross reactivity can be measured 1) using ELISA method to see if the sera from vaccinated animals or individuals will produce antibodies against species or serovars that were not used in the administered vaccine; 2) Immune cells will produce cytokines when stimulated in vitro using species or serovars that were not used in the administered vaccine. Cross protection can be measured in vitro when antibodies in sera of animals vaccinated with one species or serovars will neutralize infectivity of another chlamydia species or serovars not used in the administered vaccine.

i Bacterial Inactivation

Vaccines need to comprise some component of the target for which an immune response is desired. For examples, vaccines may comprise inactivated bacteria, particularly when the vaccine comprises whole bacteria, e.g., to ensure that the vaccine does not cause the disease or infection it is treating and/or preventing. In other words, inactivation of bacteria ensures that the vaccine does not comprise infectious particles. Approaches have included inactivation of bacteria with formalin or heat. However, formalin-inactivated vaccines have shown disease-enhancement, including showing a skewed immune response that is important to prevent disease-enhancement, and priming by mature dendritic cells, which are essential for a protective immune response. The use of live attenuated vaccines has met with limited success, as the vaccines have been shown to be minimally immunogenic.

In the methods and compositions of the invention, the nanoemulsion functions to inactivate and adjuvate the whole bacterium and/or bacterial antigens to provide a non-infectious and immunogenic bacteria vaccine. Alternatively, the bacteria (whole or antigens) can be inactivated prior to combining with the nanoemulsion. Examples of chemical methods of bacterial inactivation include, but are not limited to, formalin or β-propiolactone (β-PL), physical methods of bacterial inactivation include using heat or irradiation, or by molecular genetics means to produce a non-infectious bacterium. The simple mixing of a nanoemulsion with a vaccine candidate has been shown to produce both mucosal and system immune response.

ii Vaccine Candidates

Chlamydia immunogens that are known in the art will benefit from incorporation and formulation with the nanoemulsion of the present invention. Vaccine candidates like CT111, CT242, CT687, CT823 and CT144 (see Picard et al., High-throughput proteomic screening identifies Chlamydia trachomatis antigens that are capable of eliciting T cell and antibody responses that provide protection against vaginal challenge, VACCINE 2012: 30(29); 4387-93) are considered immunogens of the disclosed nanoemulsion chlamydia vaccine. Each of these antigens haven been shown to elicit an immunogenic response, activating CD4+ and CD8+ T cells as shown in

Table 1. Therefore, it is expected that the known adjuvant effects of the disclosed nanoemulsion would provide an additive or synergistic effect to the immune response and result in sustained protection from chlamydia infection. Accordingly, in some embodiments, the disclosed nanoemulsion chlamydia vaccines comprise at least one immunogen including CT111, CT242, CT687, CT823 and CT144.

TABLE 1

| Antigen | Protein Name and Function |
|---|---|
| CT111 | GroES - chaperonin, stress response protein (HSP10) |
| CT144 | Hypothetical protein - function unknown |
| CT242 | OmpH like - probable outer membrane protein |
| CT687 | yfhO 1 - cysteine desulfurase, amino acid metabolism |
| CT823 | htrA - serine protease |

Additional chlamydia immunogens of the present invention include, but are not limited to, surface exposed antigens isolated from the chlamydial elementary body, antigens derived from the chlamydial reticulate body inclusion membrane, and antigens derived from the chlamydial type III secretion systems. Such immunogens can be used alone in in combination to elicit protective immunity against chlamydial infection and infection-induced pathology.

For instance, recombinant major outer-membrane protein (rMOMP) has been shown to produce protective effects against chlamydia when administered in vivo. MOMP, which functions as a porin, is the principal protein at the surface of the infectious chlamydial elementary body, and therefore it is a primary candidate for anti-chlamydia vaccines. Accordingly, in some embodiments, the disclosed nanoemulsion chlamydia vaccines comprise MOMP or rMOMP.

B. Nanoemulsion

As described above, a nanoemulsion to be combined with at least one chlamydia immunogen to make a nanoemulsion chlamydia vaccine according to the invention. In one aspect, the nanoemulsion vaccine comprises an aqueous phase, at least one solvent, at least one oil, and at least one surfactant.

i Aqueous Phase

The aqueous phase can comprise any type of aqueous phase including, but not limited to, water (e.g., H2O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH2O”). In some embodiments the aqueous phase comprises phosphate buffered saline (PBS). The aqueous phase may further be sterile and pyrogen free.

ii Solvents

A nanoemulsion of the present invention is also not limited to a particular solvent, such as an organic solvent. A variety of solvents are contemplated including, but not limited to, an alcohol (e.g., including, but not limited to, methanol, ethanol, propanol, and octanol), glycerol, polyethylene glycol, and an organic phosphate based solvent.

Organic solvents in the nanoemulsion chlamydia vaccines of the invention include, but are not limited to, C1-C12 alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion chlamydia vaccine include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

iii Oil Phase

The oil in the nanoemulsion chlamydia vaccine of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

The present invention nanoemulsion is not limited to particular oil. A variety of oils are contemplated, including, but not limited to, soybean, avocado, squalene, olive, canola, corn, rapeseed, safflower, sunflower, fish, flavor, and water insoluble vitamins. Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C12-15 alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (Simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

A volatile oil of the invention can be the organic solvent, or the volatile oil can be present in addition to an organic solvent. Suitable volatile oils include, but are not limited to, a terpene, monoterpene, sesquiterpene, carminative, azulene, menthol, camphor, thuj one, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component of a disclosed nanoemulsion chlamydia vaccine is different than the oil in the oil phase.

iv Surfactants

In some embodiments, the nanoemulsion further comprises a surfactant. The present invention is not limited to a particular surfactant. A variety of surfactants are contemplated including, but not limited to, nonionic and ionic surfactants (e.g., TRITON X-100; TWEEN 20; and TYLOXAPOL).

The surfactant in the nanoemulsion chlamydia vaccine of the invention can be a pharmaceutically acceptable ionic surfactant, a pharmaceutically acceptable nonionic surfactant, a pharmaceutically acceptable cationic surfactant, a pharmaceutically acceptable anionic surfactant, a pharmaceutically acceptable zwitterionic surfactant, or any combination thereof.

In one embodiment, the nanoemulsion chlamydia vaccine comprises a cationic surfactant which is cetylpyridinium chloride (CPC). CPC may have a concentration in the nanoemulsion chlamydia vaccine of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, or greater than about 0.010%.

In a further embodiment, the nanoemulsion chlamydia vaccine comprises a non-ionic surfactant, such as a polysorbate surfactant, which may be polysorbate 80 or polysorbate 20, and may have a concentration of about 0.01% to about 5.0%, or about 0.1% to about 3% of polysorbate 80. The nanoemulsion chlamydia vaccine may further comprise at least one preservative. In another embodiment, the nanoemulsion chlamydia vaccine comprises a chelating agent.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a nonpolar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R5-(OCH2 CH2)y-OH, wherein R5 is a branched or unbranched alkyl group having from about 6 to about 22 carbon atoms and y is between about 4 and about 100, and preferably, between about 10 and about 100. Preferably, the alkoxylated alcohol is the species wherein R5 is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-100, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion chlamydia vaccine comprises a cationic surfactant, which can be cetylpyridinium chloride. In other embodiments, the nanoemulsion chlamydia vaccine comprises a cationic surfactant, and the concentration of the cationic surfactant is less than about 5.0% and greater than about 0.001%. In yet another embodiment, the nanoemulsion chlamydia vaccine comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion vaccine is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion vaccine is less than about 5.0% and greater than about 0.001%.

In another embodiment of the invention, the nanoemulsion vaccine comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the invention, the nanoemulsion vaccine comprises a cationic surfactant present in a concentration of about 0.01% to about 2%, in combination with a nonionic surfactant.

C. Additional Components of Nanoemulsion Vaccine

Additional compounds suitable for use in the nanoemulsion chlamydia vaccines of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

Suitable preservatives in the nanoemulsion chlamydia vaccines of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion chlamydia vaccine may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion chlamydia vaccine can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion chlamydia vaccine can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H2O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H2O, Ammonium formate, ≥99.0% (calc. based on dry substance, NT), Ammonium oxalate monohydrate, ≥99.5% (RT), Ammonium phosphate dibasic solution, 2.5 M in H2O, Ammonium phosphate dibasic, ≥99.0% (T), Ammonium phosphate monobasic solution, 2.5 M in H2O, Ammonium phosphate monobasic, ≥99.5% (T), Ammonium sodium phosphate dibasic tetrahydrate, ≥99.5% (NT), Ammonium sulfate solution, for molecular biology, 3.2 M in H2O, Ammonium tartrate dibasic solution, 2 M in H₂O (colorless solution at 20° C.), Ammonium tartrate dibasic, ≥99.5% (T), BES buffered saline, for molecular biology, 2× concentrate, BES, ≥99.5% (T), BES, for molecular biology, ≥99.5% (T), BICINE buffer Solution, for molecular biology, 1 M in H2O, BICINE, ≥99.5% (T), BIS-TRIS, ≥99.0% (NT), Bicarbonate buffer solution, >0.1 M Na2CO3, >0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H2O, Citric acid, anhydrous, ≥99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, ≥99.0% (T), Formic acid solution, 1.0 M in H2O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2x concentrate, HEPES, ≥99.5% (T), HEPES, for molecular biology, ≥99.5% (T), Imidazole buffer Solution, 1 M in H2O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H2O, MOPS, ≥99.5% (T), MOPS, for luminescence, ≥99.5% (T), MOPS, for molecular biology, ≥99.5% (T), Magnesium acetate solution, for molecular biology, ˜1 M in H2O, Magnesium acetate tetrahydrate, ≥99.0% (KT), Magnesium citrate tribasic nonahydrate, ≥98.0% (calc. based on dry substance, KT), Magnesium formate solution, 0.5 M in H2O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, ≥99.0% (T), Potassium acetate solution, for molecular biology, Potassium acetate solution, for molecular biology, 5 M in H2O, Potassium acetate solution, for molecular biology, ˜1 M in H2O, Potassium acetate, ≥99.0% (NT), Potassium acetate, for luminescence, ≥99.0% (NT), Potassium acetate, for molecular biology, ≥99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H2O, Potassium formate solution, 14 M in H2O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H2O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H2O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H2O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H2O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate, for molecular biology, ≥99.0% (T), Sodium phosphate dibasic dodecahydrate, ≥99.0% (T), Sodium phosphate dibasic solution, 0.5 M in H2O, Sodium phosphate dibasic, anhydrous, ≥99.5% (T), Sodium phosphate dibasic, for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate, for molecular biology, ≥99.5% (T), Sodium phosphate monobasic solution, 5 M in H2O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetateEDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H2O, Triethylammonium phosphate solution, volatile buffer, ˜1.0 M in H2O, Trimethylammonium acetate solution, volatile buffer, ˜1.0 M in H2O, Trimethylammonium phosphate solution, volatile buffer, ˜1 M in H2O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

The nanoemulsion chlamydia vaccine can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsion vaccines that may readily be diluted with water or another aqueous phase to a desired concentration without impairing their desired properties.

D. Droplet Size

The nanoemulsion chlamydia vaccine of the present invention comprises droplets having an average diameter size of less than about 1,000 nm. In other embodiments of the invention, the droplet size has an average diameter of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm.

In one embodiment, the nanoemulsion chlamydia vaccine droplets have an average diameter selected from the group consisting of less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof.

### III. Pharmaceutical Compositions

The nanoemulsion chlamydia vaccines of the invention may be formulated into pharmaceutical compositions that comprise the nanoemulsion chlamydia vaccine in a therapeutically effective amount and suitable, pharmaceutically-acceptable excipients for pharmaceutically acceptable delivery. Such excipients are well known in the art.

By the phrase “therapeutically effective amount” it is meant any amount of the nanoemulsion chlamydia vaccine that is effective in preventing, treating or ameliorating a disease or infection caused by the chlamydia pathogen associated with the immunogen administered in the composition comprising the nanoemulsion chlamydia vaccine. By “protective immune response” it is meant that the immune response is associated with prevention, treatment, or amelioration of a disease or infection associated with chlamydia. Complete prevention is not required, though is encompassed by the present invention. The immune response can be evaluated using the methods discussed herein or by any method known by a person of skill in the art.

Intranasal administration includes administration via the nose, either with or without concomitant inhalation during administration. Such administration is typically through contact by the composition comprising the nanoemulsion chlamydia vaccine with the nasal mucosa, nasal turbinates or sinus cavity. Administration by inhalation comprises intranasal administration, or may include oral inhalation. Such administration may also include contact with the oral mucosa, bronchial mucosa, and other epithelia.

Exemplary dosage forms for pharmaceutical administration are described herein. Examples include, but are not limited to, liquids, ointments, creams, emulsions, lotions, gels, bioadhesive gels, sprays, aerosols, pastes, foams, sunscreens, capsules, microcapsules, suspensions, pessary, powder, semi-solid dosage form, etc.

The pharmaceutical nanoemulsion chlamydia vaccines may be formulated for immediate release, sustained release, controlled release, delayed release, or any combinations thereof, into the epidermis or dermis. In some embodiments, the formulations may comprise a penetration-enhancing agent. Suitable penetration-enhancing agents include, but are not limited to, alcohols such as ethanol, triglycerides and aloe compositions. The amount of the penetration-enhancing agent may comprise from about 0.5% to about 40% by weight of the formulation.

The nanoemulsion chlamydia vaccines of the invention can be applied and/or delivered utilizing electrophoretic delivery/electrophoresis. Further, the composition may be a transdermal delivery system such as a patch or administered by a pressurized or pneumatic device. Such methods, which comprise applying an electrical current, are well known in the art.

The pharmaceutical nanoemulsion chlamydia vaccines for administration may be applied in a single administration or in multiple administrations. For instance, 1, 2, 3, 4, 5, 6, 7, 8, 9, or 10 administrations.

If applied topically, the nanoemulsion chlamydia vaccines may be occluded or semi-occluded. Occlusion or semi-occlusion may be performed by overlaying a bandage, polyoleofin film, article of clothing, impermeable barrier, or semi-impermeable barrier to the topical preparation.

In one embodiment, a vaccine of the invention is administered at total dosage amounts of about 20 μg/ml to about 150 μg/ml. In other embodiments, the dosage amounts can range from about 1, about 2, about 3, about 4, about 5, about 6, about 7, about 8, about 9, about 10, about 12, about 13, about 14, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 μg/ml. In another embodiment, a vaccine of the invention is administered at total doses of about 4 μg/ml.

In another embodiment, a vaccine of the invention is administered such that therapeutically effective antibody titer levels are maintained for at least about 4 months, about 5 months, about 6 months, about 7 months, about 8 months, about 9 months, about 10 months, about 11 months, about 12 months, or about 1, about 2, about 3, about 4, about 5, about 6, about 7, about 8, about 9, or about 10 years.

In some embodiments embodiment, a vaccine of the invention is administered as a pharmaceutical composition. In one embodiment, the pharmaceutical composition comprising a vaccine of the invention can be formulated in a wide variety of oral or parenteral dosage forms on clinical application. Each of the dosage forms can contain various disintegrating agents, surfactants, fillers, thickeners, binders, diluents such as wetting agents or other pharmaceutically acceptable excipients.

A nanoemulsion vaccine composition can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, and a suspension. Further, the composition may be a controlled release formulation, sustained release formulation, immediate release formulation, or any combination thereof. Further, the composition may be a transdermal delivery system.

In another embodiment, the pharmaceutical composition comprising a vaccine of the invention can be formulated in a solid dosage form for oral administration, and the solid dosage form can be powders, granules, capsules, tablets or pills. In yet another embodiment, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In a further embodiment, the pharmaceutical composition comprising a vaccine of the invention can be formulated as a sublingual or buccal dosage form. Such dosage forms comprise sublingual tablets or solution compositions that are administered under the tongue and buccal tablets that are placed between the cheek and gum.

In yet a further embodiment, the pharmaceutical composition comprising a vaccine of the invention can be formulated as a nasal dosage form. Such dosage forms of the present invention comprise solution, suspension, and gel compositions for nasal delivery.

In one embodiment, the pharmaceutical composition can be formulated in a liquid dosage form for oral administration, such as suspensions, emulsions or syrups. In other embodiments, the liquid dosage form can include, in addition to commonly used simple diluents such as water and liquid paraffin, various excipients such as humectants, sweeteners, aromatics or preservatives. In particular embodiments, the composition comprising udenafil or a pharmaceutically acceptable salt thereof can be formulated to be suitable for administration to a pediatric patient.

In one embodiment, the pharmaceutical composition can be formulated in a dosage form for parenteral administration, such as sterile aqueous solutions, suspensions, emulsions, non-aqueous solutions or suppositories. In other embodiments, the non-aqueous solutions or suspensions can include propyleneglycol, polyethyleneglycol, vegetable oils such as olive oil or injectable esters such as ethyl oleate. As a base for suppositories, witepsol, macrogol, tween 61, cacao oil, laurin oil or glycerinated gelatin can be used.

The dosage of the pharmaceutical composition can vary depending on the patient's weight, age, gender, administration time and mode, excretion rate, and the severity of disease.

An exemplary nanoemulsion adjuvant composition according to the invention is designated “W805EC” adjuvant. The composition of W805EC adjuvant is shown in Table 2 below. The mean droplet size for the W805EC adjuvant is ˜400 nm. All of the components of the nanoemulsion are included on the FDA inactive ingredient list for Approved Drug Products.

TABLE 2
W₈₀5EC Formulation

| | W₈₀5EC-Adjuvant |
|---|---|
| Function | Mean Droplet Size ≈ 400 nm |
| Aqueous Diluent | Purified Water, USP |
| Hydrophobic Oil (Core) | Soybean Oil, USP (super refined) |
| Organic Solvent | Dehydrated Alcohol, USP (anhydrous ethanol) |
| Surfactant | Polysorbate 80, NF |
| Emulsifying Agent | Cetylpyridinium Chloride, USP |
| Preservative | |

The nanoemulsion adjuvants are formed by emulsification of an oil, purified water, nonionic detergent, organic solvent and surfactant, such as a cationic surfactant. An exemplary specific nanoemulsion adjuvant is designated as “60% W₈₀5 EC”. The 60% W₈₀5 EC-adjuvant is composed of the ingredients shown in Table 3 below: purified water, USP; soybean oil USP; Dehydrated Alcohol, USP [anhydrous ethanol]; Polysorbate 80, NF and cetylpyridinium chloride, USP (CPCAll components of this exemplary nanoemulsion are included on the FDA list of approved inactive ingredients for Approved Drug Products.

TABLE 3
Composition of 60% W₈₀5EC-Adjuvant (w/w %)

| Ingredients | 60% W₈₀5EC |
|---|---|
| Purified Water, USP | 54.10% |
| Soybean Oil, USP | 37.67% |
| Dehydrated Alcohol, USP (anhydrous ethanol) | 4.04% |
| Polysorbate 80, NF | 3.55% |
| Cetylpyridinium Chloride, USP | 0.64% |

### IV. Stability of Nanoemulsion Vaccines of the Invention

The nanoemulsion chlamydia vaccines of the invention can be stable at about 40° C. and about 75% relative humidity for a time period of at least up to about 2 days, about 3 days, about 4 days, about 5 days, about 10 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, or at least up to about 12 months.

In another embodiment of the invention, the nanoemulsion chlamydia vaccines of the invention can be stable at about 25° C. and about 60% relative humidity for a time period of at least up least up to about 2 days, about 3 days, about 4 days, about 5 days, about 10 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, or at least up to about 18 months.

In another embodiment, the nanoemulsion chlamydia vaccines of the invention can be stable at about 4° C. for a time period of at least up to about 2 days, about 3 days, about 4 days, about 5 days, about 10 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 1 year, about 1.5 years, or about 2 years.

In another embodiment, the nanoemulsion chlamydia vaccines of the invention can be stable at about −20° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

These stability parameters are applicable to nanoemulsion adjuvants and/or nanoemulsion chlamydia vaccines.

### V. Methods of Manufacture

The nanoemulsions of the invention can be formed using classic emulsion forming techniques. See e.g., U.S. 2004/0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water or PBS. The nanoemulsions of the invention are stable, and do not deteriorate even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should kill or disable chlamydia bacteria to a detectable level, or induce a protective immune response to a detectable level. The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

In an exemplary method of the invention for preparing a nanoemulsion chlamydia vaccine useful for the treatment or prevention of an chlamydia infection in humans, the method comprises: (a) synthesizing in a prokaryotic host one or more full length or immunogenic fragment of chlamydia antigens utilizing recombinant DNA genetics vectors and constructs, wherein the chlamydia antigen is selected from the group consisting of major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, and CT144; (b) isolating the one or more antigens or immunogenic fragments thereof from the chlamydia; and (c) formulating the one or more antigens with an oil-in-water nanoemulsion. In another embodiment of the invention, the method comprises (a) obtaining isolated whole chlamydia bacteria; and (b) formulating the chlamydia bacteria with an oil-in-water nanoemulsion. In yet another embodiment, both whole chlamydia bacteria and isolated chlamydia antigens can be utilized in the nanoemulsion chlamydia vaccines of the invention.

These nanoemulsion-containing containers can further be packaged with instructions for use to form kits.

The invention is further described below by reference to the examples, which are provided for illustration only. The invention is not limited to the examples, but rather includes all variations that are evident from the teachings provided herein. All publicly available documents referenced herein, including but not limited to U.S. patents, are specifically incorporated by reference.

### VI. Definitions

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

“A treatment” is intended to target the disease state and combat it, i.e., ameliorate or prevent a disease or infection associated with chlamydia. The particular treatment thus will depend on the disease state to be targeted and the current or future state of medicinal therapies and therapeutic approaches. A treatment may have associated toxicities.

The terms “administration of” or “administering” an active agent or vaccine of the invention should be understood to mean providing the agent or vaccine of the invention to a subject in need of treatment by a means that can be introduced into that individual's body in a therapeutically useful form and therapeutically effective amount.

The term “therapeutically effective amount” refers to a sufficient quantity of the vaccine of the present invention, in a suitable composition, and in a suitable dosage form to treat or prevent the symptoms, progression, or onset of the disease or complications seen in subjects who have a chlamydia infection or may become exposed to chlamydia. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

The term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

The terms “individual,” “subject,” and “patient” are used interchangeably herein. As used herein, a subject or the like may refer to a human or an animal. An animal subject can include, but is not limited to, cats, dogs, horses, pigs, sheep, cattle, goats, hamsters, gerbils, ferrets, mice, rats, lizards, snakes, turtles, and koala bears.

The term “nanoemulsion” as used herein, includes small oil-in-water dispersions or droplets, as well as other lipid structures which can form as a result of hydrophobic forces which drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present invention contemplates that one skilled in the art will appreciate this distinction when necessary for understanding the specific embodiments herein disclosed. Nanoemulsion particle size generally varies from 300 to 600 nanometers.

As used herein, the term “antigen” refers to proteins, glycoproteins or derivatives or fragment that can contain one or more epitopes (linear, conformation, sequential, T-cell) which can elicit an immune response. Antigens can be separated in isolated bacterial proteins or peptide derivatives.

As used herein, the term “isolated” refers to bacterial, proteins, glycoproteins, peptide derivatives or fragment or polynucleotide that is independent from its natural location. Bacterial components that are independently obtained through recombinant genetics means typically leads to products that are relatively purified.

As used herein, the term “adjuvant” refers to an agent that increases the immune response to an antigen (e.g., a chlamydia antigen). As used herein, the term “immune response” refers to a subject's (e.g., a human or another animal) response by the immune system to immunogens (i.e., antigens) the subject's immune system recognizes as foreign. Immune responses include both cell-mediated immune responses (responses mediated by antigen-specific T cells and non-specific cells of the immune system—Th1, Th2, Th17, and IFN-γ) and humoral immune responses (responses mediated by antibodies). The term “immune response” encompasses both the initial “innate immune responses” to an immunogen (e.g., a chlamydia antigen) as well as memory responses that are a result of “acquired immunity.”

As used herein, the term “immune enhancing” refers to a significant boost in the level and breath of the innate and acquired immune response to a given pathogen following administration of a vaccine of the present invention relative to the level of innate and acquired immunity when a vaccine of the present invention has not been administered.

As used herein, the term “chlamydia whole bacteria” refers to native, recombinant, and mutant whole chlamydia, including all known species and subtypes.

As used herein, the term “chlamydia antigens” refers to proteins, glycoproteins and peptide fragments that are generally derived from the extracellular surface of chlamydia bacteria, although they may also be recombinant or synthetic. Preferred chlamydia antigens are major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, CT144, and fusions, derivatives, or fragments thereof. Chlamydia antigens are generally extracted from bacterial isolates from infected cell cultures, or produced synthetically, or by using recombinant DNA methods. The chlamydia antigens can be modified by chemical, genetic, or enzymatic means resulting in variants, fusion proteins, peptides, or fragments.

As used herein, the term “multivalent vaccines” refers to a vaccine comprising more than one antigenic determinant of a single bacterial agent or multiples species or serovars. As used herein, multivalent vaccine comprise chlamydia whole bacteria and/or multiple chlamydia antigens. Multivalent vaccines can be constructed with antigens derived from all known species and subtypes of chlamydia.

As used herein, the term “subunit” refers to isolated and generally purified chlamydia peptides or glycoproteins that are individually or mixed further with nanoemulsion comprising a vaccine composition. The subunit vaccine composition is free from whole or mature bacteria, cells, or lysate of cells or bacteria. The method of obtaining a bacterial antigens that is included in a subunit vaccine can be conducted using standard recombinant genetics techniques and synthetic methods and with standard purification protocols.

The following examples are given to illustrate the present invention. It should be understood, however, that the invention is not to be limited to the specific conditions or details described in these examples. All printed publications referenced herein are specifically incorporated by reference.

### EXAMPLES

### Example 1—Nanoemulsion Preparation

The purpose of this example was to describe preparation of a nanoemulsion to be used in a nanoemulsion chlamydia vaccine.

To manufacture the nanoemulsion, the water soluble ingredients are first dissolved in water. The soybean oil is then added and the mixture is mixed using high shear homogenization and/or microfluidization until a viscous white emulsion is formed. The emulsion may be further diluted with water to yield the desired concentration of emulsion or cationic surfactant.

The nanoemulsion (NE) composition was formulated according to Table 4.

TABLE 4
Nanoemulsion composition

| Component | Concentration v/v |
|---|---|
| Water | 84.7% |
| Soybean Oil | 12.6% |
| Ethanol | 1.35% |
| Polysorbate 80 | 1.18% |
| Cetylpyridinium chloride (CPC) | 0.2% |

The nanoemulsion can then be combined with one or more chlamydia immunogens to form a nanoemulsion chlamydia vaccine according to the invention.

### Example 2—Vaccine Adjuvant

The purpose of this example is to describe exemplary nanoemulsions useful as adjuvants for a chlamydia vaccine.

A total of 10 nanoemulsion formulations were prepared: W₈₀5 EC alone, six W₈₀5 EC+Poloxamer 407 and Poloxamer 188 (P407 and P188) formulations as well as two W₈₀5 EC+Chitosan and one W₈₀5 EC+Glucan formulation have been produced and assessed for stability over 2 weeks under accelerated conditions at 40° C. (Table 5). All 10 nanoemulsions were stable for at least 2 weeks at 40° C.

TABLE 5
W₈₀5EC Formulations

| | | Method of | Particle | Zeta | |
|---|---|---|---|---|---|
| Nanoemulsion | Ratios: | Addition of | Size | Potential | |
| (lot) | CPC:Tween:Poloxamer | Poloxamer | (nm) | (mV) | pH |
| W₈₀5EC | 1:6 | — | 450 | 60 | 4.9 |
| W₈₀5EC + 3% P407 | 1:6 | External | 500 | 56 | 5.9 |
| W₈₀5EC/P407 | 1:5:1 | Internal | 391 | 46 | 5.5 |
| W₈₀5EC/P407 | 1:1:5 | Internal | 253 | 36 | 5.2 |
| W₈₀5EC/P188 | 1:5:1 | Internal | 526 | 54 | 5.1 |
| W₈₀5EC/P188 | 1:3:3 | Internal | 416 | 54 | 5.7 |
| W₈₀5EC/P188 | 1:1:5 | Internal | 370 | 47 | 5.2 |
| W₈₀5EC + 0.3% Chitosan LMW | 1:6 | External | 505 | 60 | 5.7 |
| W₈₀5EC + 0.3% Chitosan MMW | 1:6 | External | 523 | 60 | 5.4 |
| W₈₀5EC + 0.03% β(1,3) Glucan | 1:6 | External | 491 | 41 | 6.3 |

The following formulations are exemplary nanoemulsions useful in the chlamydia vaccines of the invention: (1) Formulation 1, W₈₀5 EC (NE80), comprising: (a) CPC/Tween 80 (ratio of 1:6), and (b) Particle size ˜500 nm (Table 6); and Formulation 2, W₈₀P₁₈₈5 EC (NE188), comprising: (a) CPC/Tween 80/P188 (ratio of 1:1:5), (b) Particle size ˜300 nm (Table 7).

TABLE 6
Formulation 1
Composition of 60% W₈₀5EC adjuvant

| Ingredient | w/w % |
|---|---|
| Distilled water | 54.1 |
| CPC | 0.64 |
| Tween 80 | 3.55 |
| Ethanol | 4.04 |
| Soybean oil | 37.7 |

TABLE 7
Formulation 2
Composition of 60% W₈₀P₁₈₈5EC adjuvant

| Ingredient | w/w % |
|---|---|
| Distilled water | 54.1 |
| CPC | 0.64 |
| Tween 80 | 0.6 |
| Poloxamer 188 | 3 |
| Ethanol | 4.03 |
| Soybean oil | 37.7 |

### Example 3—Formulation Results

The purpose of this example was to demonstrate the associated of a nanoemulsion with bacterial antigen.

Transmission Electron Micrographs and Sectioning Technique:

Twenty mL of the nanoemulsion adjuvant alone or with Fluzone® was fixed with 1% (w/v) osmium tetroxide solution. The fixed preparations were mixed with histogel in 1:10 ratio to form a solid mass. The solid mixture of was sliced into thin 1 mm slices and rinsed with double distilled deionizer water. The cross-sectioned samples were dehydrated with ascending concentrations (30%, 50%, 70%, 90%, 100%) of component A of the Durcupan® kit (Fluka, EM #14020) in double distilled deionizer water. These samples were transferred into embedding solution (mixture of components A, B, C and D) of the Durcupan® kit. The embedded samples were sectioned to a 75 nm thickness and placed on 300 mesh carbon-coated copper grid. The sections on the grids were stained with saturated uranyl acetate in distilled and deionizer water (pH 7) for 10 minutes followed by lead citrate for 5 minutes. The samples were viewed with a Philips CM-100 TEM equipped with a computer controlled compustage, a high resolution (2K x 2K) digital camera and digitally imaged and captured using X-Stream imaging software (SEM Tech Solutions, Inc., North Billerica, Mass.).

Electron Micrographs:

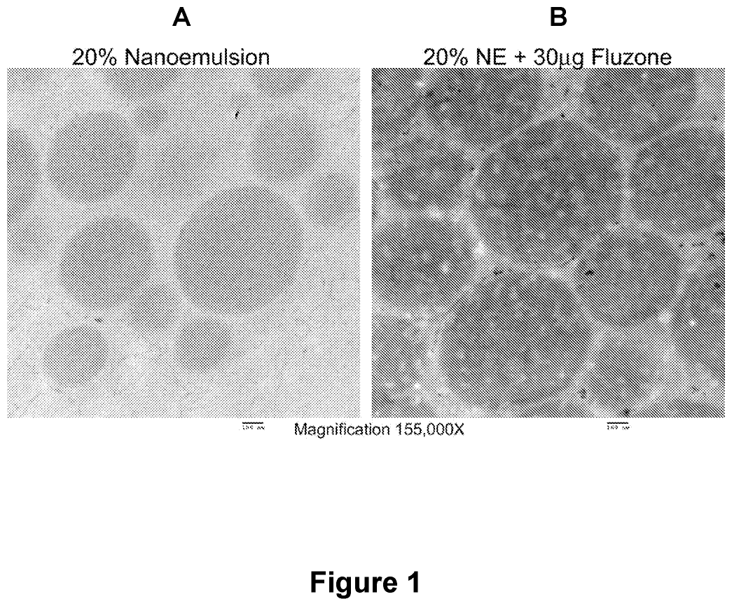

Cross sectioned TEM of 20% W₈₀SEC nanoemulsion showed nanoemulsion droplets with a uniform inner core material. Nanoemulsion vaccine containing 30 μg of HA shows discrete antigen materials/particles inside the oil core of the droplets that represent the Fluzone® antigens. Since the antigen is incorporated in the core, and is surrounded by the core material, it is protected from staining by the electron dense stain. This leads to a white counter staining effect in the core. The localization of the antigen within the core shields the antigen-sensitive protein subunits in the emulsion, and may protect the antigen from degradation, and thus enhancing stability. There are very few Fluzone® particles outside of the NE particles that were stained dark in color (FIGS. 1A and 1B).

### Example 4—Prevention of Chlamydia Infection in a Subject

This example demonstrates the use of the disclosed chlamydia nanoemulsion vaccine in the prevention of chlamydia infection in a subject. In this example, at least one bacterial antigen associated with chlamydia is formulated in the disclosed nanoemulsion for the purposes of a vaccine.

Subjects suspected of having an increased risk of contracting chlamydia receive at least one administration of a prophylactically effective amount of a disclosed chlamydia nanoemulsion vaccine, either alone or in combination with one or more additional agents for the treatment or prevention of chlamydia. The disclosed chlamydia nanoemulsion vaccine and/or additional agents are administered orally, intravaginally, intranasally, intrathecally, intraocularly, intradermally, transmucosally, iontophoretically, topically, systemically, intravenously, subcutaneously, intraperitoneally, or intramuscularly according to methods known in the art. Subjects will be evaluated daily for the presence and/or severity of signs and symptoms associated with chlamydia infection, including, but not limited to, e.g., burning feeling during urination, discharge from the penis or vagina, pain in the lower abdomen, painful sexual intercourse in women, pain in the testicles in men, itching or burning sensations around the genitals, and/or odor. Treatments may be maintained until such a time as one or more signs or symptoms of chlamydia infection are prevented.

It is predicted that subjects receiving a prophylactically effective amounts of a disclosed chlamydia nanoemulsion vaccine, will have a reduced or abolished risk of contracting chlamydia. It is further expected that administration of disclosed chlamydia nanoemulsion vaccine in combination with one or more additional agents will have additive or synergistic effects in this regard.

These results will show that disclosed chlamydia nanoemulsion vaccine are useful in the prevention of chlamydia or other infectious disease. Accordingly, a disclosed chlamydia nanoemulsion vaccine is useful in methods comprising administering the disclosed chlamydia nanoemulsion vaccine to a subject in need thereof for the prevention or treatment of chlamydia or another infectious disease.

While certain of the preferred embodiments of the present invention have been described and specifically exemplified above, it is not intended that the invention be limited to such embodiments. Various modifications may be made thereto without departing from the scope and spirit of the present invention.

1. A vaccine composition comprising:
(a) an immune enhancing nanoemulsion, wherein the nanoemulsion comprises an oil-in-water nanoemulsion or a dilution thereof; and
(b) at least one chlamydia bacteria antigen, wherein the chlamydia bacteria antigen is whole chlamydia, an isolated chlamydia antigen, a recombinant chlamydia antigen, or a combination thereof, and wherein the chlamydia bacteria antigen is present within the nanoemulsion.

2. The vaccine composition of claim 1, wherein the chlamydia bacteria antigen is inactivated prior to incorporation into a nanoemulsion and/or where the chlamydia antigen is inactivated by the nanoemulsion.

3. The vaccine composition of claim 1, wherein the chlamydia bacteria antigen is derived from chlamydia bacteria and comprises at least one major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, or CT144 or an immunogenic fragment thereof.

4. The vaccine composition of claim 1, wherein one or more chlamydia antigens further comprise nucleotide modifications denoting attenuating phenotypes.

5. The vaccine composition of claim 1, wherein at least one chlamydia antigen is present in a fusion protein.

6. The vaccine composition of claim 1, wherein at least one chlamydia antigen is present in an immunogenic peptide fragment of major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, or CT144 or an immunogenic derivative thereof.

7. The vaccine composition of claim 1, wherein the immune enhancing nanoemulsion is capable of inducing Th1, Th2, Th17, and/or IFN-γ immune responses.

8. The vaccine composition of claim 1, wherein the nanoemulsion particle size is from about 300 nm up to about 600 nm.

9. The vaccine composition of claim 1, further comprising:
(a) an adjuvant; and/or
(b) a pharmaceutically acceptable carrier.

10. The vaccine composition of claim 1, wherein:
(a) the vaccine composition is formulated for administration via a method selected from the group consisting of parenterally, intravaginally, orally and intranasally; and/or
(b) the vaccine composition is formulated for parenteral administration, which is subcutaneous, intraperitoneal or intramuscular injection.

11. (canceled)

12. A method for preparing a nanoemulsion chlamydia bacteria vaccine useful for the treatment or prevention of an chlamydia infection in a subject comprising:
(a) synthesizing in a prokaryotic host one or more full length or immunogenic fragment chlamydia bacteria antigens utilizing recombinant DNA genetics vectors and constructs, wherein the chlamydia bacteria antigen is selected from the group consisting of major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, and CT144;
(b) isolating the one or more antigens or immunogenic fragments thereof from the prokaryotic host; and
(c) formulating the one or more antigens with an oil-in-water nanoemulsion.

13. The method according to claim 12, wherein the chlamydia bacteria is C. trachomatis.

14. A subunit vaccine composition comprising an immune enhancing nanoemulsion combined with one or more chlamydia bacteria antigens, wherein the nanoemulsion further comprises an oil-in-water nanoemulsion or a dilution thereof and isolated viral antigens preferentially comprised within the nanoemulsion.

15. The subunit vaccine composition of claim 14, wherein the one or more chlamydia antigens are derived from chlamydia bacteria and comprise isolated major outer-membrane protein (MOMP), chlamydial outer-membrane complex (COMC), polymorphic outer-membrane proteins (POMPs or Pmps), the 60 and 75 kDa heat-shock proteins, the type-III secretory system structural proteins, exoglycolipid, Inc proteins, Cap1, CT111, CT242, CT687, CT823, or CT144 or an immunogenic fragment thereof.

16. The subunit vaccine composition of claim 14, further comprising:
(a) an adjuvant; and/or
(b) at least one pharmaceutically acceptable carrier.

17. The subunit vaccine composition of claim 14, wherein the vaccine composition is formulated for administration either parenterally, orally, intravaginally, or intranasally.

18. A method of treating a subject in need comprising administering to the subject a nanoemulsion chlamydia bacteria vaccine composition, wherein following administration the vaccine induces an immune response against infection caused by chlamydia bacteria in the subject, and wherein the vaccine composition comprises:
(a) an immune enhancing nanoemulsion, wherein the nanoemulsion comprises an oil-in-water nanoemulsion or a dilution thereof; and
(b) at least one chlamydia bacteria antigen, wherein the chlamydia bacteria antigen is whole chlamydia, an isolated chlamydia antigen, a recombinant chlamydia antigen, or a combination thereof, and wherein the chlamydia bacteria antigen is present within the nanoemulsion.
